(19) United States
(12) Patent Application Publication
(10) Pub. No.: US 2025/0275851 A1
(43) Pub. Date: Sep. 4, 2025

(54) ANCHOR POSITION VERIFICATION FOR PROSTHETIC CARDIAC VALVE DEVICES

(71) Applicant: Shifamed Holdings, LLC, Campbell, CA (US)

(72) Inventor: Amr SALAHIEH, Saratoga, CA (US)

(21) Appl. No.: 18/979,182

(22) Filed: Dec. 12, 2024

Related U.S. Application Data
(63) Continuation of application No. 17/655,978, filed on Mar. 22, 2022, now Pat. No. 12,201,521.
(60) Provisional application No. 63/164,514, filed on Mar. 22, 2021.

(51) Int. Cl. A61F 2/24; A61M 5/00

(52) U.S. Cl. CPC A61F 2/2418; A61M 5/007; A61F 2220/0008; A61F 2250/0098; A61M 2210/125

(57) ABSTRACT

A device for treating a diseased native valve in a patient is provided, the device including a frame structure and a plurality of leaflets. The device can further include a spiral anchor configured to extend around an outer circumference of the frame structure. The anchor can be configured to deliver a contrast agent into or near a target tissue. Other embodiments and methods of use are also provided.

### CROSS REFERENCE TO RELATED APPLICATIONS

This application is a continuation application of U.S. application Ser. No. 17/655,978, filed Mar. 22, 2022, which claims the benefit of U.S. Provisional Application No. 63/164,514, filed Mar. 22, 2021, which are herein incorporated by reference in its entirety.

### BACKGROUND

Blood flow between heart chambers is regulated by native valves—the mitral valve, the aortic valve, the pulmonary valve, and the tricuspid valve. Each of these valves is a passive one-way valve that opens and closes in response to differential pressures. Patients with valvular disease have abnormal anatomy and/or function of at least one valve. For example, a valve may suffer from insufficiency, also referred to as regurgitation, when the valve does not fully close, thereby allowing blood to flow retrograde. Valve stenosis can cause a valve to fail to open properly. Other diseases may also lead to dysfunction of the valves.

The mitral valve, for example, sits between the left atrium and the left ventricle and, when functioning properly, allows blood to flow from the left atrium to the left ventricle while preventing backflow or regurgitation in the reverse direction. Native valve leaflets of a diseased mitral valve, however, do not fully prolapse, causing the patient to experience regurgitation.

While medications may be used to treat diseased native valves, the defective valve often needs to be repaired or replaced at some point during the patient's lifetime. Existing prosthetic valves and surgical repair and/or replacement procedures may have increased risks, limited lifespans, and/or are highly invasive. Some less invasive transcatheter options are available, but most are not ideal. A major limitation of existing transcatheter mitral valve devices, for example, is that the mitral valve devices are too large in diameter to be delivered transseptally, requiring transapical access instead. Furthermore, existing mitral valve replacement devices are not optimized with respect to strength-weight ratio and often take up too much space within the valve chambers, resulting in obstruction of outflow from the ventricle into the aorta and/or thrombosis.

At times, fixation of a valve replacement device to the native anatomy is achieved using an anchor structure that is delivered transcatheter. It can be a challenge for clinicians to confirm that the anchor is properly positioned with respect to the native anatomy during implantation, e.g., due to the size and materials used to construct anchors that are suitable for minimally invasive implantation.

Thus, a new valve device that overcomes some or all of these deficiencies is desired.

### SUMMARY OF THE DISCLOSURE

A prosthesis for treating a diseased native valve is provided, the prosthesis comprising a frame structure having a plurality of leaflets therein, and a spiral anchor configured to extend around an outer perimeter of the frame structure, comprising a wall defining a lumen, and at least one port in fluid communication with the lumen, the lumen and the at least one port shaped and sized to transport and deliver a contrast agent that is detectable by a visualization modality.

In additional embodiments, the wall defining the lumen is an interior wall within an outer perimeter of the spiral anchor.

In other embodiments, the wall defining the lumen is continuous from a proximal end to a distal end of the spiral anchor.

In some embodiments, the wall defining the lumen is an exterior wall along an outer perimeter of the spiral anchor (e.g., a monorail).

In some embodiments, a proximal end of the wall is configured to fluidly couple with a distal end of a contrast agent delivery catheter, during the delivery of the prosthesis.

In some embodiments, the at least one port is at a distal tip of the anchor.

In additional embodiments, the at least one port is proximal to a distal tip of the anchor.

In some embodiments, the at least one port is oriented toward a leaflet and/or an annulus of the diseased native valve, when the spiral anchor is near a delivery position with respect to the diseased native valve.

In other embodiments, the delivery position is a sub-annular space of the diseased native valve.

In some embodiments, the contrast agent comprises barium-sulfate, iodine, or an iodine-based material.

A method of delivering a valve prosthesis is provided, comprising advancing a distal end of a delivery device to a first side of a native valve, deploying an anchor from a delivery configuration to a deployed configuration on the first side of the native valve, the anchor comprising at least one port for delivering a contrast agent therefrom, advancing the anchor in the deployed configuration from the first side of the native valve to a second side of the native valve, rotating the anchor in the deployed configuration around one or more structures on the second side of the native valve, delivering the contrast agent through the at least one port, identifying a characteristic of the contrast agent in an image to confirm that the anchor has been rotated around the one or more structures.

In additional embodiments, the method includes releasing the anchor from the distal end of the delivery device.

In some embodiments, delivering the contrast agent comprises delivering into a blood flow path of the heart.

In some embodiments, delivering the contrast agent comprises delivering following the step of advancing to the second side of the native valve.

In other embodiments, the characteristic of the contrast agent comprises an extent of dispersion.

In some embodiments, confirming that the anchor has been fully rotated comprises confirming that dispersion of the contrast agent is substantially confined to a selected region.

In some embodiments, the selected region comprises a sub-annular space of the native valve.

In some embodiments, the method further comprises repeating at least one of the rotating, delivering, and identifying steps until the extent of dispersion is within the selected region.

In other embodiments, the image comprises a fluoroscopic image.

In additional embodiments, the anchor comprises the anchor of claim 1.

### BRIEF DESCRIPTION OF THE DRAWINGS

FIGS. 1A-1B show an exemplary valve prosthesis (also referred to herein as “valve device”) for replacement of a valve, such as a mitral valve.

FIGS. 2A-2H show sequential views of an exemplary method of implanting a valve prosthesis.

FIGS. 3A-3C illustrate one embodiment of an anchor that can include one or more structures that are adapted to transport and deliver a contrast agent.

FIGS. 4A-4B illustrate visualization of a contrast agent delivered by an anchor.

FIG. 5 is a flowchart describing a method of delivering contrast agent with an anchor of a valve replacement device.

### DETAILED DESCRIPTION

Described herein are systems, devices, or methods for treatment or replacement of a diseased native valve of the heart, for example a mitral valve.

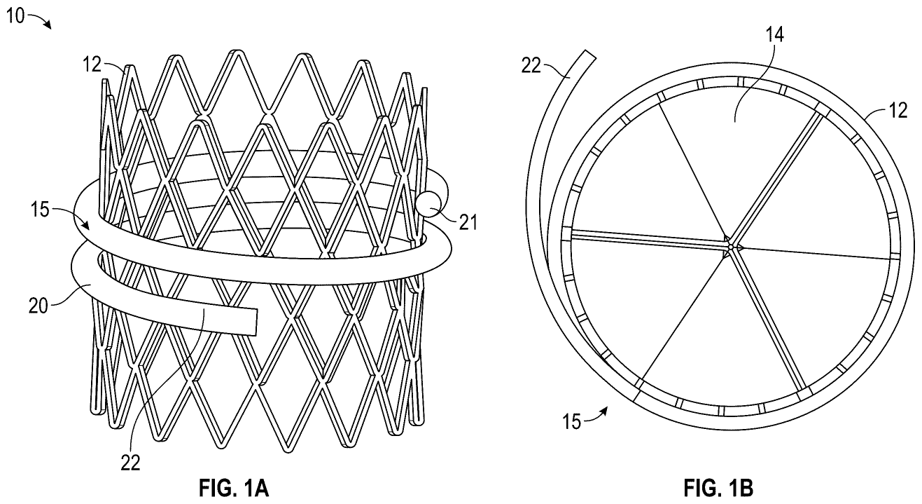

FIGS. 1A-1B show an exemplary valve prosthesis 10 (also referred to herein as “valve device”) for replacement of a valve, such as a mitral valve. The illustrated valve prosthesis 10 comprises a frame structure 12, leaflets 14, and an anchor 15. The anchor 15 includes a wire 20 formed in a spiral shape around the frame structure 12.

The exemplary frame structure 12 is configured like a stent. The frame structure 12 has an expanded state and an unexpanded (e.g., collapsed or compressed) state. The compressed state is sized and dimensioned for percutaneous insertion and the expanded state is sized and dimensioned for implantation in a native valve of a patient, such as a mitral valve.

The anchor 15 can include a spiral member, such as wire 20, having a proximal end 21 and a distal end 22. The anchor 15 can be configured to engage with the frame structure 12 via a compression fit. The wire 20 can be formed of a material having sufficient rigidity to hold a predetermined shape. In an exemplary embodiment, the wire 20 can be formed of a shape memory material (e.g. NiTi). Further, the anchor 15 prior to implantation may comprise a flat spiral shape such that loops of the anchor are generally positioned within the same plane (the plane being perpendicular to a longitudinal axis of a delivery device). Additionally, in some embodiments, the distal end 22 can be rounded and/or atraumatic.

The valve prosthesis 10 can be configured for replacing a mitral valve with the distal end 22 configured for insertion through a commissure.

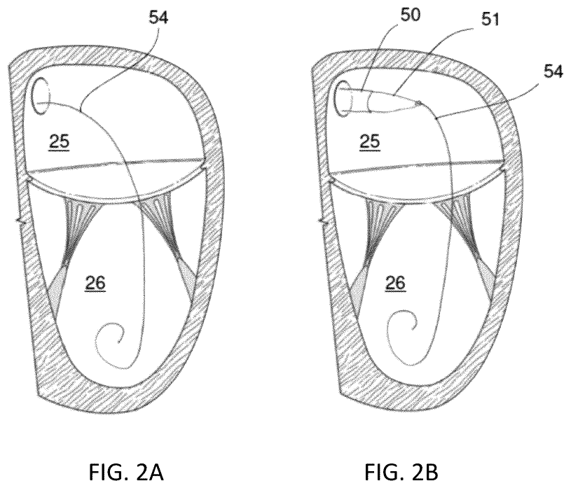

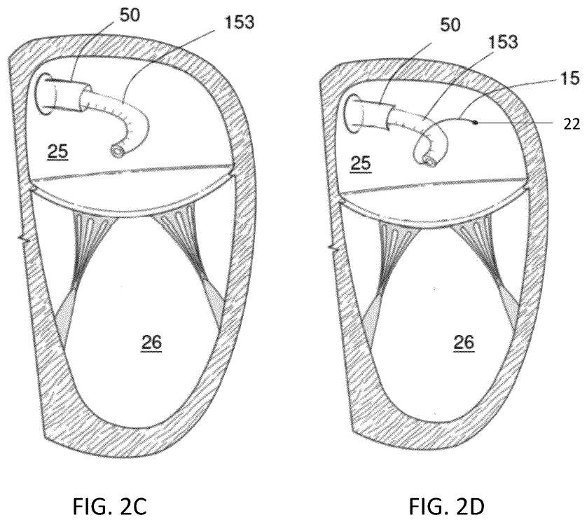

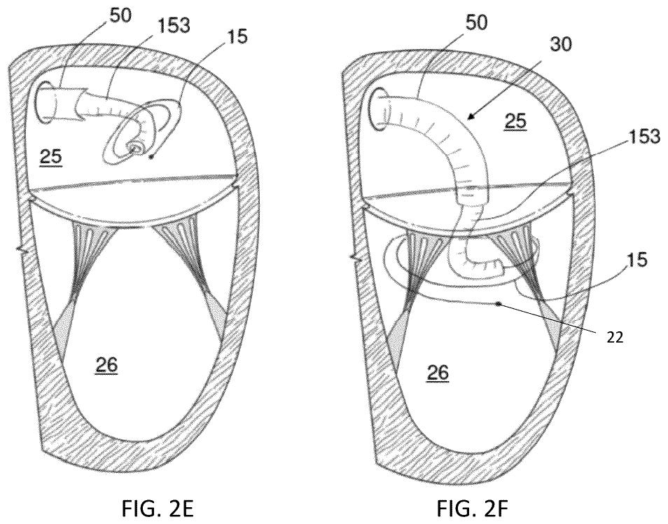

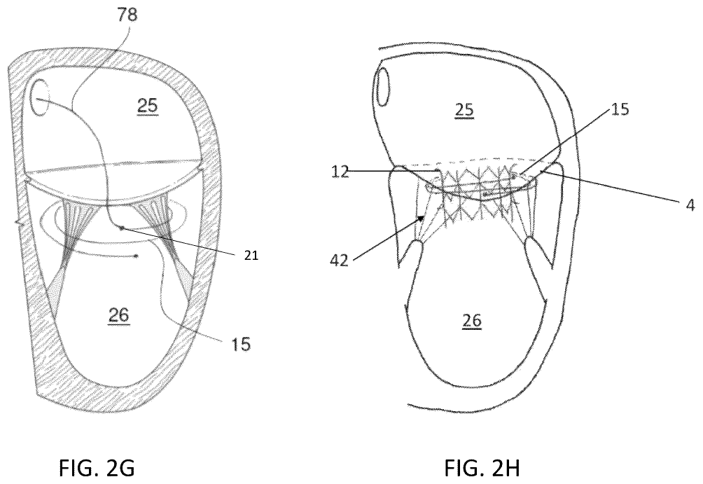

FIGS. 2A-2H show sequential views of an exemplary method of implanting a valve prosthesis 10. At FIG. 2A, a transseptal puncture is made. A guidewire 54 is then routed through the puncture site and left either in the left atrium 25 or across the mitral valve into the left ventricle 26. At FIG. 2B, the outer sheath 50 (optionally with an inner dilator 51) is tracked over the guidewire 54 until the distal end of the outer sheath 50 protrudes into the left atrium 25. The guidewire 54 and inner dilator 51 are then removed from the outer sheath 50. At FIG. 2C, an inner shaft and attached distal anchor guide 153 are inserted through the outer sheath 50 until the distal tip of the anchor guide 153 extends into the left atrium 25. The anchor guide 153 can be positioned and/or oriented as desired by steering the distal end of the sheath 50 and/or rotating the inner shaft and anchor guide 153 relative to the sheath 50. At FIG. 2D, once the anchor guide 153 is in the correct orientation, the anchor 15 can be pushed out through distal tip of the anchor guide 153 (with the distal tip 22 extending out of the guide 153 first). At FIG. 2E, the anchor 15 can fully deploy into the atrium 25. At FIG. 2F, the entire delivery system 30 can be pushed and steered (for example, via steering mechanisms in the outer sheath 50) towards an apex of the ventricle 26, crossing through the mitral valve. In some embodiments, counter-rotation of the anchor 15 may aid in getting the anchor 15 across the mitral valve without tangling. Once the anchor 15 is at the correct depth within the ventricle 26, forward rotation of the anchor 15 (via forward rotation of the inner shaft and guide 153) will allow the anchor 15 to encircle the mitral leaflets and chordae (i.e., with the distal end 22 leading the encircling). At FIG. 2G, the outer sheath 40, inner sheath, and anchor guide 153 are removed, leaving a tether 78 in place (and attached to the proximal end 21 of the anchor 15). Next, the frame structure 12 can then be delivered over the tether 78 and into place within the anchor 15. At FIG. 2H, the frame structure 12 has been delivered, the tether 78 has been released from the proximal end 21 of the anchor 15 to leave the prosthesis 10 in place in the mitral valve 4. As shown in the exemplary FIG. 2H, the anchor 15 is positioned to encircle substantially all of the chordae 42 and is “high” in the ventricle 26. An anchor 15 that has a high position can be adjacent the inferior surface of the annulus of mitral valve 4.

In some embodiments, an anchor 15 is adapted to transport and deliver a biocompatible contrast agent that can enhance an imaging modality image during and/or following delivery of the anchor. The imaging modality can be any modality that is compatible with minimally invasive procedures, such as fluoroscopy and/or echocardiography. Examples of contrast agents (e.g., media) comprise iodine, iodine-based compounds, barium-sulfate, or saline. Without being bound by theory, regarding an x-ray imagining modality, the contrast agent can block or limit the passage of x-rays therethrough. Regarding an ultrasound imaging modality, the contrast agent may possess an increased echogenicity. An anchor 15 that delivers such a contrast agent during and/or following its deployment can alter the appearance of the heart anatomy and/or of the circulation therein, for example of one or more chambers or vessels of the heart.

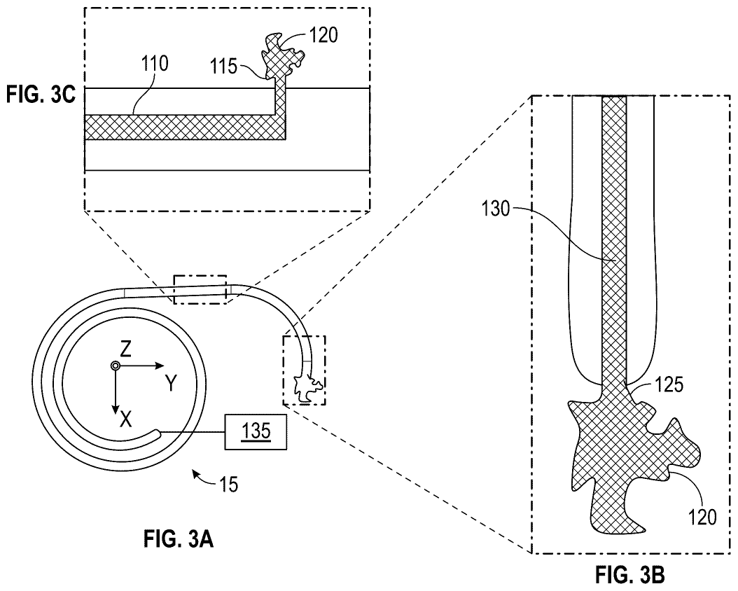

Referring to FIGS. 3A-3C, in some embodiments, the anchor 15 can include one or more structures that are adapted to transport and deliver a contrast agent 120 from a contrast agent source 135 through the anchor and into or near a target tissue or anatomy of the patient (such as a heart). FIG. 3A depicts an entirety of the anchor 15, including a distal tip (further illustrated in FIG. 3B) and portions of the anchor proximal to the tip (further illustrated in FIG. 3C). It should be noted that in the illustrated embodiment, the distal portion of the anchor 15 can have a larger radius of curvature compared to other, more proximal portions of the anchor, causing the distal portion to extend or “stick out” from the rest of the anchor. It should be understood that other embodiments of the anchor may not have this distal portion that extends outwards, and can instead comprise an anchor that has only a single radius of curvature (such as the anchor depicted in FIG. 1A).

The contrast agent source 135 can be a vessel, container, or volume either disposed within the anchor 15 or remote from the anchor. The contrast agent source 135 is fluidly coupled with the lumen 130 of the anchor. In some embodiments, the contrast agent source can be a syringe exterior to the anchor and to the patient. In other embodiments, the lumen of the anchor can be fluidly coupled to another lumen within a delivery catheter, and the contrast agent source can be either fluidly coupled to the delivery catheter lumen or remote from (but fluidly coupled to) the delivery catheter. The contrast agent source can further include a mechanism for delivering contrast agent from the source into the lumen(s) and out through the port(s) of the anchor. For example, in one embodiment the contrast agent can be delivered by deploying a syringe. In other embodiments, pumps or other ways of pressurizing and/or creating a flow of the contrast agent can be implemented.

As depicted in the example FIG. 3B, in some embodiments, the contrast agent 120 is delivered from a tip of the anchor 15, which comprises a lumen 130 and one or more ports 125. In some embodiments, the lumen 130 is formed by an inner wall of the anchor 15 that is located within an outer perimeter of the anchor (e.g., is substantially centrally located). In some embodiments, the lumen 130 is formed by an outer wall of the anchor 15 (e.g., as a monorail). The lumen 130 can traverse from a proximal portion to a distal portion of the anchor 15. In some embodiments that comprise a monorail construction, the lumen may traverse less than the entirety of the length of the anchor 15. For example, a proximal end of the lumen 130 may terminate distal to a proximal end of the anchor 15, and/or a distal end of the lumen 130 may terminate proximal to a distal end of the anchor 15.

As depicted in the example FIG. 3C, in some embodiments, the contrast agent 120 is delivered from one or more portions of the anchor 15 that are proximal to the tip, where the proximal portions include a lumen 110 and one or more ports 115. In some embodiments, the one or more ports 115 are positioned on the body of the anchor 15 such that, when delivered in a selected orientation and/or position in the heart, the one or more ports 115 are at least partially obstructed by one or more portions of the native heart. For example, an obstruction can comprise a leaflet of the native valve, one or more chordae, an inferior surface of the valve annulus, or a portion of the ventricular heart wall. In some embodiments, the one or more ports 115 are oriented to be generally radially-outward, generally radially-inward, and/or generally along a superior aspect of the anchor 15 (e.g., superior when implanted in the ventricle of the heart).

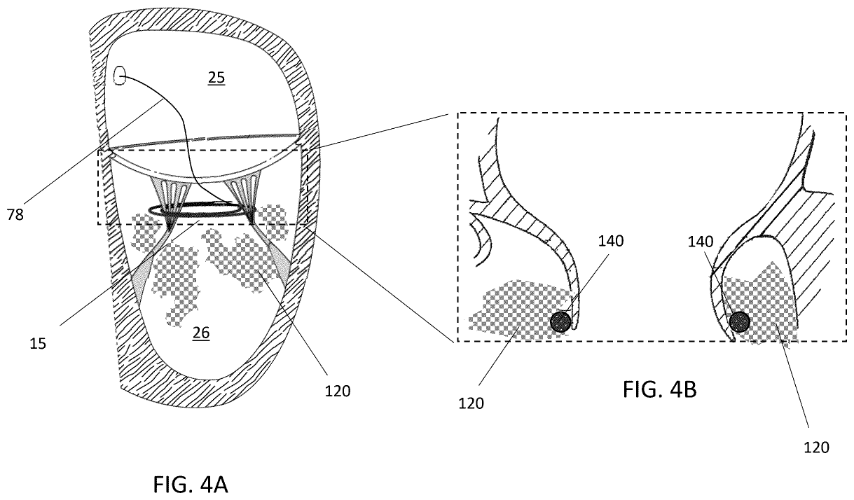

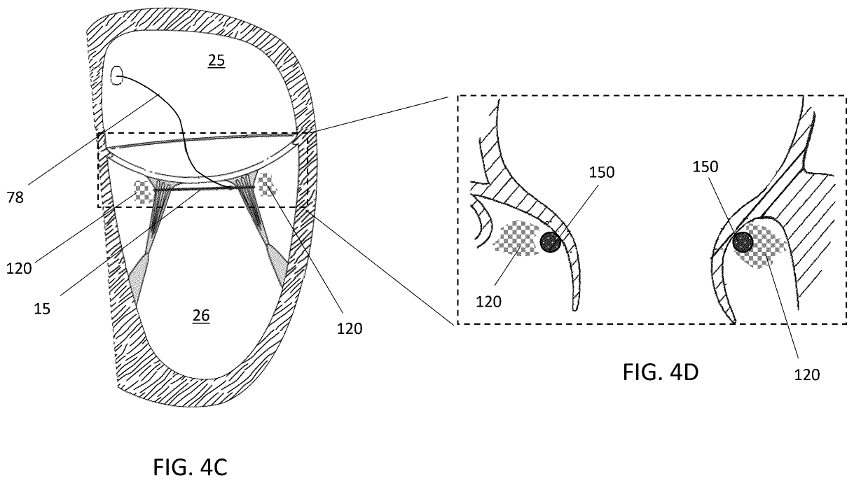

In some embodiments, feedback regarding an orientation and/or position of the anchor 15 with respect to the native heart anatomy can be provided according to one or more characteristics of the blood flow, visualized in the presence of the contrast agent that is delivered via the anchor 15. Within a ventricle of the heart, blood flow velocity is often reduced in a region that is inferior and peripheral to the valve annulus. In contrast, blood flow velocity within the ventricle is increased within a central region of the chamber, moving toward the apex of the heart. Referring now to FIGS. 4A-4B, in some embodiments an anchor 15 that is position “low” with respect to the sub-annular tissue (e.g., anchor 140 seen in cross-section, FIG. 4B) may release contrast agent 120 into a space having a relatively high blood flow velocity, such that the contrast agent 120 dilutes and/or disperses relatively quickly. The contrast agent 120 may appear to have a reduced or diminished intensity in such a condition, and/or to occupy a greater region of space within the heart. Referring now to FIGS. 4C-D, in some embodiments, an anchor 15 that is positioned in a preferred “high” near the sub-annular tissue (e.g., anchor 150 seen in cross-section, FIG. 4D) may release contrast agent 120 into a space having a relatively low blood flow velocity, such that the contrast agent 120 gathers in the space with little dilution and/or dispersion. The contrast agent 120 may appear to have a greater intensity with the given imaging modality in such a condition. In combination with an appropriate imaging modality, the position of the anchor 15 can be (e.g., indirectly) measured or confirmed by the intensity and/or dispersion of a contrast agent 120.

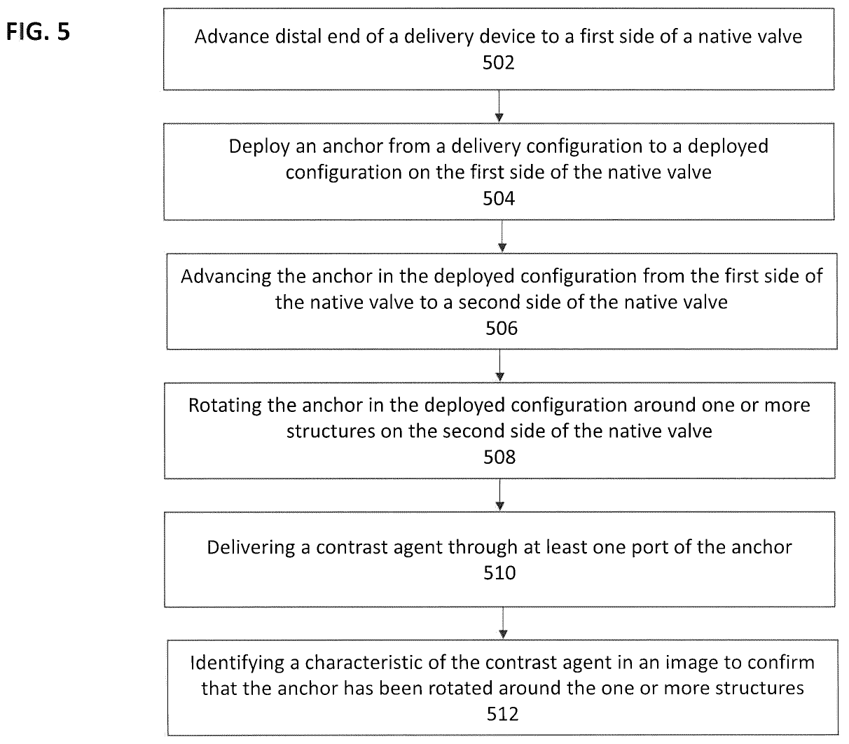

FIG. 5 is a flowchart that describes a method of delivering a valve prosthesis including delivering a contrast agent with an anchor of a valve prosthesis. In some embodiments, at step 502, the method can include advancing a distal end of a delivery device to a first side of a native valve (FIG. 2C). At step 504, the method can further include deploying an anchor from a delivery configuration to a deployed configuration on the first side of the native valve (FIG. 2D-FIG. 2E). In some embodiments, the anchor can comprise at least one port for delivering a contrast agent therefrom. At step 506, the method can further include advancing the anchor in the deployed configuration from the first side of the native valve to a second side of the native valve and at step 508, rotating the anchor in the deployed configuration around one or more structures on the second side of the native valve (FIG. 2F). At step 510, the method can include delivering the contrast agent through the at least one port (as shown in FIGS. 3A-3C). Finally, the method can include identifying a characteristic of the contrast agent in an image to confirm that the anchor has been rotated around the one or more structures.

In some embodiments, the method of FIG. 5 can further include releasing the anchor from the distal end of the delivery device.

In some embodiments, delivering the contrast agent comprises delivering into a blood flow path of the heart. In other embodiments, delivering the contrast agent comprises delivering following the step of advancing to the second side of the native valve.

in some examples, the characteristic of the contrast agent comprises an extent of dispersion. In one implementation, confirming that the anchor has been fully rotated comprises confirming that dispersion of the contrast agent is substantially confined to a selected region.

In some examples, the selected region comprises a sub-annular space of the native valve.

In some implementations of the method, the method further includes repeating at least one of the rotating, delivering, and identifying steps until the extent of dispersion is within the selected region.

In some examples, the image comprises a fluoroscopic image.

In other embodiments, the anchor comprises any of the anchors described in this disclosure.

Additional elements of valve prostheses, anchors, and methods of delivery are described in PCT Application No. PCT/US2019/047542 filed on Aug. 21, 2019, PCT Application No. PCT/US2019/057082 filed on Mar. 19, 2019, PCT Application No. PCT/US2019/068088 filed on Dec. 20, 2019, and PCT Application No. PCT/US2020/23671, the entireties of which are incorporated by reference herein in their entireties.

It should be understood that any feature described herein with respect to one embodiment can be substituted for or combined with any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. (canceled)

2. A device for treating a diseased native valve, the device comprising:
a spiral anchor configured to encircle one or more chordae in a subject's heart, the spiral anchor having a lumen disposed therein and one or more ports;
a contrast agent source in fluid communication with the lumen and the one or more ports and being configured to deliver a contrast agent that is detectable by a visualization modality into a blood flow path of the subject's heart.

3. The device of claim 2, wherein the spiral anchor is configured to engage with a prosthetic valve device via a compression fit.

4. The device of claim 2, further comprising a wall defining the lumen that is an interior wall within an outer perimeter of the spiral anchor.

5. The device of claim 2, wherein the lumen is continuous from a proximal end to a distal end of the spiral anchor.

6. The device of claim 2, wherein the contrast agent source is disposed within the anchor.

7. The device of claim 2, wherein the contrast agent source is remote from the anchor.

8. The device of claim 7, wherein a proximal end of the lumen is configured to fluidly couple with a distal end of a contrast agent delivery catheter.

9. The device of claim 2, wherein the one or more ports is at a distal tip of the anchor.

10. The device of claim 2, wherein the one or more ports is proximal to a distal tip of the anchor.

11. The device of claim 2, wherein the one or more ports is oriented toward a leaflet and/or an annulus of the diseased native valve, when the spiral anchor is near a delivery position with respect to the diseased native valve.

12. The device of claim 11, wherein the delivery position is a sub-annular space of the diseased native valve.

13. The device of claim 2, wherein the contrast agent comprises barium-sulfate, iodine, or an iodine-based material.

14. A method of delivering a valve prosthesis, comprising:
advancing a distal end of a delivery device to native valve;
rotating a spiral anchor around one or more chordae of the native valve;
delivering a contrast agent through at least one port of the spiral anchor into a blood flow path of the heart; and
obtaining an image of the contrast agent within the heart.

15. The method of claim 14, further comprising confirming that the anchor has been rotated around the one or more chordae.

16. The method of claim 14, further comprising confirming proper positioning of the spiral anchor based on the image.

17. The method of claim 14, further comprising releasing the spiral anchor from the distal end of the delivery device.

18. The method of claim 14, wherein the image comprises a fluoroscopic image.
